(19) United States
(12) Patent Application Publication
(10) Pub. No.: US 2008/0108913 A1
(43) Pub. Date: May 8, 2008

(54) SMART APPARATUS FOR GAIT MONITORING AND FALL PREVENTION

(75) Inventors: Corinne S. Lengsfeld, Denver, CO (US); Rahmat A. Shoureshi, Golden, CO (US)

Correspondence Address: GREENLEE WINNER AND SULLIVAN P C, 4875 PEARL EAST CIRCLE, SUITE 200, BOULDER, CO, 80301, US

(73) Assignee: Colorado Seminary, which owns and operates The University of Denver, Denver, CO (US)

(21) Appl. No.: 11/556,858

(22) Filed: Nov. 6, 2006

(51) Int. Cl. A61B 5/103

(52) U.S. Cl. 600/595

(57) ABSTRACT

Provided is a system for monitoring gait. More particularly, the system comprises: one or more pressure sensors; an algorithm which compares the data from the pressure sensor(s) to a stability profile, and provides a feedback value; means for communicating the feedback value; and a power source. Also provided is a method for gait analysis comprising: collecting signals from one or more pressure sensors located in pressure proximity to a foot, generating a test profile; comparing the test profile to a stability profile; generating a feedback signal; and communicating the feedback signal. The system may also comprise one or more accelerometers.

### BACKGROUND OF THE INVENTION

Between 2011 and 2030 the demographics of the United States will dramatically change as the baby boomers retire. The percentage of people over age 65 will shift from 12% to 20% nationwide [Federal Interagency Forum on Aging Related Statistics, Key Indicators of Well-Being]. Coupled with this rising population is the knowledge that the greatest reduction in quality of life and highest percentage of health care costs are associated with individuals older than 85 years of age. Since 1950, the development of early detection and assistive technologies has improved the quality of life and life expectancy for seniors in the United States. Examples include: hip replacement numbers have risen well beyond 250,000 and the average age of death for men and women of all races has risen by 4.2 years or 5% [National Vital Statistics, 2002]. Further improvements must concentrate on accelerating these advances as well as reducing the associated costs. New sensors and computer technologies place these goals within reach.

21^{st} century advances in the care of the elderly will utilize embedded technology within a care delivery and prevention plan. Consequently, the technology must assist the patient with evaluating and building on their own strengths, while decreasing the time and labor commitments of caregivers. Data collection must be held to a minimum to maintain sensitivity while monitors/sensors used for data collection need to be small, non-intrusive, and affordable.

Diminished stability in older adults poses a serious health risk resulting from fall events. Approximately 28% of people between 70-79 years of age, 47% of people between 80-90 years of age and 65% of people between 90-99 years of age die from injuries related to falling [National Safety Council, 2000]. More than one-third of adults over 65 years of age report at least one fall per year, and more than 10% of these falls lead to serious injury. In 1994, fall related medical expenses in the aging population exceeded $20 billion, and this is anticipated to reach $32 billion by 2020. The prevention of falls in the aging population has become a leading health issue in America.

Skin, bone and muscle injuries do not completely account for the decline in mobility observed after a fall. More than 70% of women and 26% of men over the age of 60 who have experienced a fall report a fear of falling that leads to a significant increase in balance or gait disorders. Following a fall, these individuals reduce their activity out of fear, further increasing their risk of a fall and hampering their rate of recovery, as one day in bed translates into 7 to 14 new days of physical therapy to regain the same degree of mobility. Recent studies indicate that older adults who fell during a given 12-month period were 50% more likely to die compared to cohorts who did not fall regardless if the fall event resulted in injury or not.

Diminished balance is frequently a multi-factorial result of aging (e.g. visual, auditory and tactile sensory deficits, reduced reaction times and muscular strength, and progressive alteration in sense of joint-position), disease (e.g. acute illness, hypertension, stroke, arthritis, etc.), medications and/or environmental factors (e.g., lighting, slippery surfaces, obstacles, etc.). General models for balance suggest that when balance is perturbed, a person controls his/her center of mass by repositioning the center of pressure. While environmental factors typically initiate the perturbation in balance, it is the reduction in strength, cognition and sensory input that yield insufficient compensations that result in falls.

Many current interventions focus solely on strength or balance issues inherent to the multifaceted problem of falling. Traditional programs include exercise programs, adoption of assistive walking tools, and/or protection such as hip protectors. Proprioceptive physical exercises (e.g. yoga or soft gymnastics) has demonstrated the largest benefit to postural control, followed by bioenergetic activities such as jogging or cycling; while little improvement appears in subjects simply participating in a walking program. Walking aids such as canes and walkers increase patient confidence and reduce fall frequency, but the devices are frequently undesirable to patients due to personal preference or environmental limitations. While fall protection devices appear to reduce fall related fractures under certain circumstances, they do not address patient fears or mortality rates associated with non-injured fallers.

There have been some attempts to monitor physical characteristics that may be related to falls. US application 2003/0009308 describes gyro sensors and force-sensitive resistors mounted in an insole to measure acceleration and rotation of the insole. This device is reported to determine the cadence and ankle power of the user, which may be useful to diagnose disorders in which ankle-push off is reduced. U.S. Pat. No. 5,357,696 describes a force-concentrating system which directs all forces in a shoe to a central point. This total force is compared to a desired amount of force and a notification is communicated to the user if the total force is higher or lower than desired. This device is reported to be useful in recuperation following an injury or surgery. U.S. Pat. No. 5,678,448 describes a multiplicity of force sensors covering the entire area of the user's foot. An alarm sounds when a force is greater than a predetermined threshold force. U.S. Pat. No. 5,408,873 describes measuring the compressive force exerted by a foot using an insole having a plurality of layers of dielectric material. U.S. Pat. No. 6,033,370 describes a capacitive force sensor having a plurality of layers of dielectric and conductive materials. U.S. Pat. No. 5,323,650 describes a force sensor for use in a shoe, where sensors are arranged in a pattern that covers the entire area of the foot. U.S. Pat. No. 5,566,479 describes a shoe or insole having a cutout region where a force sensing resistor is placed. When the pressure on the force sensing resistor exceeds a threshold value, an alarm sounds.

The reported systems do not prevent falls or allow the elderly or injured to build muscle strength and coordination. There is a need for such a system. In addition, there is a need for a system that can detect changes in walking gait speed and cycle to predict illnesses, for example.

### BRIEF SUMMARY OF THE INVENTION

Provided is a system for monitoring gait. More particularly, the system comprises: one or more sensors; means for capturing the data from the sensor(s); means for generating a feedback value from a comparison of the data from the sensor(s) and a stability profile; and means for communicating the feedback value. The means for generating a feedback value can comprise an algorithm. The system can also comprise a power source. Also provided is a system for monitoring gait comprising: a plurality of sensors which generate a signal; a circuit means electrically connected to the plurality of sensors whereby said signal is collected; a transmission means to transmit the signals; a power source electrically connected to said plurality of sensors, circuit means, and transmission means; software that receives the transmitted signal and compares the transmitted signal to a stability profile and generates a feedback signal; and a feedback means which transmits the feedback signal. Also provided is a method for gait analysis comprising: collecting signals from one or more sensors, generating a test profile; comparing the test profile to a stability profile; generating a feedback signal; and communicating the feedback signal.

In the embodiments described herein, the sensors comprise one or more pressure sensors and optionally, one or more accelerometers. Accelerometers provide additional information about gait speed, stride length, and gait timing. Accelerometers are an optional component of the embodiments described herein.

Accelerometers may also be used as a redundancy to check the data received from the pressure sensors. In particular embodiments, one, two, three or more accelerometers may be used

In one embodiment, the sensor(s) are located in a shoe, shoe insole, or sock. As used herein, “shoe” indicates a device which at least partially encloses the foot. A shoe may contain attachment devices known in the art such as velcro, laces, or elastic, or other attachment devices known in the art or may be attached to the foot by the use of tape, for example medical tape. As used herein, “shoe insole” indicates a structure that may be placed in a shoe, such as a conventional insole known in the art. A shoe insole may also be placed on the foot and attached using any suitable means, such as the use of tape, string, or elastic bands. The use of a separate insole without a shoe may be useful if the patient is unable to be fitted with shoes.

The pressure sensors may be any suitable pressure sensor, as known in the art. One suitable example is the FlexiForce, obtained by Teskan, South Boston, Mass. There are other pressure sensors that are useful in the invention. A combination of pressure sensors may be used. In one embodiment, there is a plurality of pressure sensors. In one embodiment, the pressure sensors are located in pressure communication with different parts of the foot. The different parts of the foot may include one or more of: big toe pad, heel, under one or more metatarsals, inner ball, outer ball, outside edge. The pressure sensors may be different sizes, depending on the location or other factors, as known in the art. There may be as many or as few pressure sensors as desired to obtain the desired sensitivity of measurement, as balanced by cost, durability and other factors as known in the art. In different embodiments, there are one, two, three, four, five, six, seven, eight, nine or ten pressure sensors. In one embodiment, there are more than ten pressure sensors. In one embodiment, there are less than ten pressure sensors. In one embodiment, there are less than five pressure sensors. In one embodiment, there are five or fewer pressure sensors. In one embodiment, there are more than two pressure sensors. All individual values and ranges are intended to be included to the extent as if they were individually listed. In one embodiment, the pressure sensors do not cover a substantial portion of the user's foot. In one embodiment, the pressure sensors are not arranged in an array.

Suitable accelerometers and the use thereof is known to one of ordinary skill in the art without undue experimentation.

The system can be powered by any suitable energy source. The power source can be one or more of: kinetic energy (energy generated by the user walking); and alternating or direct current, including one or more batteries which may be rechargeable or non-rechargeable. In one embodiment, there is a combination of energy sources used. Different portions of the system can be powered in different ways. For example, the portions of the system that are present in the shoe, shoe insole or sock may be powered by kinetic energy, while the other portions of the system are powered by alternating current. Alternatively, the portions of the system that are present in the shoe, shoe insole or sock may be powered by batteries. In a portable system, it is desired that no parts of the system require wall current.

In one embodiment, the means for communicating the feedback value is selected from the group consisting of: visual indication, tactile indication, audible indication and combinations thereof. Visual indication can include different colored lights which correspond to various feedback conditions. For example: green can be used to indicate the situation is safe, yellow can be used to indicate the situation requires caution, and red can be used to indicate the situation is unsafe and the behavior should be stopped. These lights may be present in any suitable reporting device. For example, the lights may be incorporated in eyeglasses which the user may wear. The lights may be incorporated in a hand-held device or a device worn around the neck. The lights may be incorporated in a wall-mounted system, for example, in a physician's office or patient room. Audible indication can include different tones and/or volumes of tones to correspond to various feedback conditions. Tactile indication can include a physical sensation presented to the user if a particular feedback condition is present. For example, a system that presents a signal such as a tapping motion can be incorporated in a band worn on a body part such as the wrist or arm, and the system can be designed to send a signal when an unsafe condition is present.

The invention is useful for any animal or person that applies pressure to one or more feet. The invention is useful for mammals. The invention is useful for humans. The invention is also useful for animals, including horses, cows or dogs, where the alteration in gait can be used as an early determiner of illness or injury.

The system can be used in different ways. For example, the system can be used to prevent falls. In this example, the pressure sensors generate a profile of the center of mass of the individual (“test profile”). This profile is compared to an ideal center of mass profile “stability profile” using an algorithm such as a Neuro-Fuzzy decision-making system which uses a learning algorithm to determine its rules by processing data samples. An inference engine that integrates advantages of a neural network and fuzzy logic is incorporated in this system.

This neuro-fuzzy inference engine has five layers, in one embodiment, and can be used for any number of inputs and outputs (MIMO). It employs the gradient descent method and the least square estimation (LSE) algorithms to train the network. FIG. 5 shows the architecture of the inference engine.

Layer 1: (Fuzzification layer) Each node generates a membership degree of a linguistic value. The k^{th} node in this layer performs the following operation:

$${O}_{k}^{1} = {μ}_{{A}_{ij}} ( {x}_{i} ) = \frac{1}{1 + {( \frac{{x}_{i} - {a}_{ij}}{{b}_{ij}} )}^{2}} \qquad ( 8 )$$

Layer 2: (Multiplication Layer) Each node calculates the firing strength of each rule by using multiplication operation.

$$\begin{matrix}{O}_{k}^{2} = {\prod }_{i} {O}_{ij}^{1} ( {x}_{i} ) & ( 1 \leq k \leq 4 )\end{matrix} \qquad ( 9 )$$

Layer 3: (Normalization layer) The number of nodes in this layer is the same as the first layer, where the output of layer two is determined according to:

$$\begin{matrix}{O}_{k}^{3} = \frac{{O}_{k}^{2}}{{\sum }_{k} {O}_{k}^{2}} & ( 1 \leq k \leq 4 )\end{matrix} \qquad ( 10 )$$

Layer 4: (Defuzzification layer) The number of nodes in this layer is equal to the number of nodes in layer one times the number of outputs. The defuzzified value for the

$$\begin{matrix}{y}_{k} = \{ \begin{matrix}{c}_{k} - {d}_{k} \sqrt{\frac{1}{{O}_{k}^{3}} - 1} & if k = odd \\ {c}_{k} + {d}_{k} \sqrt{\frac{1}{{O}_{k}^{3}} - 1} & if k = even\end{matrix} \} & ( 1 \leq k \leq 4 )\end{matrix} \qquad ( 11 )$$

where {cₖ,dₖ} are consequent parameters and are used to adjust the shape of the membership function of the consequent part. Then, the output of this layer becomes:

$${O}_{k}^{4} = {O}_{k}^{3} \cdot {y}_{k} = \begin{matrix}\{ \begin{matrix}{O}_{k}^{3} \cdot ( {c}_{k} - {d}_{k} \sqrt{\frac{1}{{O}_{k}^{3}} - 1} ) & if k = odd \\ {O}_{k}^{3} \cdot ( {c}_{k} + {d}_{k} \sqrt{\frac{1}{{O}_{k}^{3}} - 1} ) & if k = even\end{matrix} \} & ( 1 \leq k \leq 4 )\end{matrix} \qquad ( 12 )$$

Layer 5: (Summation layer) Here, the number of nodes is equal to the number of outputs. There is only one connection between each node in layer three and a node in the output layer:

$$\begin{matrix}{O}_{1}^{5} = {\sum }_{k} {O}_{k}^{4} & ( 1 \leq k \leq 4 )\end{matrix} \qquad ( 13 )$$

In the training process, the engine tries to find the minimizing error function between target value and the network output. For a given training data set with P entries, the error function is defined as:

$$\begin{matrix}E = {\sum }_{p = 1}^{P} {E}_{p} = \frac{1}{2} {\sum }_{p = 1}^{P} {( {T}_{p} - {O}_{1 , p}^{5} )}^{2} , & ( 1 \leq p \leq P ) .\end{matrix} \qquad ( 14 )$$

There are several key attributes of this neuro-fuzzy inference engine that adapt it well for the present invention:

(a) it uses a combination of a fuzzy inference engine and an adaptive neural network

(b) it uses fuzzy reasoning for both fuzzification and defuzzification, that is, the membership functions are half of a bell-shape function called monotonic nonlinear functions

(c) it can be applicable to Multi-input and Multi-output (MIMO) system

(d) it uses associated hybrid learning algorithm to tune the parameters of membership functions: Feedforward Process; Least Square Estimation; Backward Process; Gradient Descent method

(e) it uses an optimal learning rate that is updated after each learning process

(f) it has the least number of coefficient to learn, has a fast convergence rate, and is therefore suitable for real-time applications.

This inference engine can be used in modeling and mapping of uncertain systems whose mathematical representation (e.g. differential equations) is not available to predict its future behavior. It integrates the best features of a fuzzy system (fuzzy reasoning) and neural networks (learning). Neuro-fuzzy inference technique provides a means for the fuzzy modeling to learn information about a data set, which will compute and generate the membership function parameters, so that the associated fuzzy inference system can track the given input and output pattern. Its learning method works similarly to that of neural networks. This network can be used to find out system parameters and unknown factors through the training process, which means it achieves the goal of system identification.

The algorithm provides a feedback value. The feedback value can be used in many different ways. In one embodiment, the feedback can be communicated to the user. In one example of this embodiment, the user is notified if the test profile is within the stability profile parameters or outside the stability profile parameters. The notification can be visual, audio and/or vibratory feedback, as described elsewhere herein. The user is altered if his behavior is “safe” (little risk of falling) or unsafe (high risk of falling). The user can thus continue his behavior without concern for falling if the behavior is safe, or change his behavior in response to the notification. The system can also be used to detect changes in walking gait speed and cycle that are predictors of illnesses or measures of reactions to changes in a patient's drug regimen, for example, by using accelerometers. The system detects changes in gait speed and cycle and provides feedback to either the user or a care-giver, for example. “On-demand” physical therapy can be performed using the system. In this aspect of the invention, the user can build stability and coordination by correlating how changes in movement change the feedback. The feedback and/or data from the pressure sensor(s) may be stored on electronic media for future use. This can be useful for medical professionals to review and monitor a patient's activity for use in a physical therapy protocol, for example. There are other uses of the invention which will become apparent upon review of the disclosure herein. These uses are intended to be encompassed here.

### BRIEF DESCRIPTION OF THE FIGURES

FIG. 1 shows a block diagram of one embodiment of the invention.

FIG. 2 shows a flow chart showing one embodiment of the system (not to scale). 1 is one or more force/pressure and accelerator sensors. 2 is a controller. 3 is software. 4 is a circuit. 5 is the feedback. 6 is a receiver/transmitter. 7 is a power source. Although the insole, microcontroller, and output feedback are shown separately in FIG. 2, all components could be present on the same apparatus, such as the insole.

FIG. 3 shows one example of the system incorporated in a shoe.

FIG. 4 shows real-time data collected wirelessly from insole sensor system: (i) postural sway when balancing on a single foot, (ii) force data from a single foot during normal walking.

FIG. 5 shows an architecture of the inference engine.

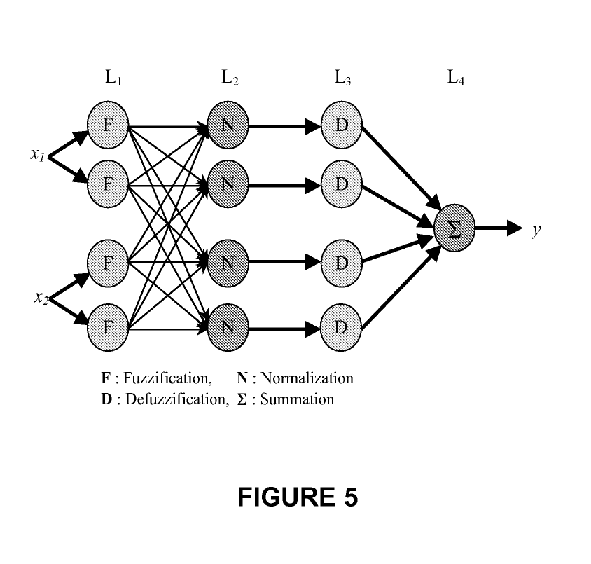

### DETAILED DESCRIPTION OF THE INVENTION

The following description contains non-limiting examples which are intended to further illustrate some embodiments of the invention.

This invention augments the patient's diminished natural sensory feedback system, and provides information to the patient on their current stability situation such as stable [green], therapeutic [yellow] and danger [red] zones. Stability information allows individuals to assess their own performance and regain confidence in their ability to remain upright after a perturbation. By intentionally moving oneself into the therapeutic zone of instability, a patient can use this system to perform their own strength and coordination building physical therapy. Embedding this technology into existing physical therapy programs monitored and designed by rehabilitation specialists, patients gain access to individualized, interactive physical therapy programs on-demand, 24 hours a day, thus extending the period of active therapy and reducing the time to acquire (or reacquire) improved stability. Physicians also gain access to data on daily balance control and conditioning. Artificial intelligence (neuro-fuzzy logic) provides for automated stability zone narrowing or widening as the patient abilities change with time. Such a device is also useful to evaluate the effects of a new therapy (such as a new medication or a change in dose) on patient stability. This is useful, for example, when a potentially destabilizing (centrally active) medication is added to an existing regimen.

It is known that a healthy, stress-free work environment improves productivity through improved concentration. Illness, whether mental or physical, can decrease our action functions, particularly those controlled by our sensory system. This decrease in function may be very subtle in young adults who can divide and refocus their attention efficiently, but the elderly, especially those individuals with borderline or dependent functionality confined to an assisted living or nursing home environment, for example, may exhibit a dramatic decline in motor function during illness or stress. Gait is regulated by the basal ganglia using information provided by the prefrontal and/or frontal cortices. Therefore, although walking is a previously learned motor program, older adults experience a reduced walking speed while performing a dual cognitive task, such as talking. Because illness (or other stressors such as new medications) can impair executive functions, the onset of a physical illness in older adults may first become detectable as a subtle change in gait speed or timing cycle. Fried (1991) suggested that an unperceived decline in physical function precedes clinically observable declines. This “preclinical disability” arises because daily function must decrease dramatically before older individuals will recognize a problem through self-evaluation and seek medical help. Therefore, longitudinal gait variability can be used as an early warning indicator for caregivers to detect illnesses earlier, especially in those individuals who have difficulty articulating their health state. Caregivers might also gain faster feedback on drug interactions and therapeutic performance before a patient's condition declines.

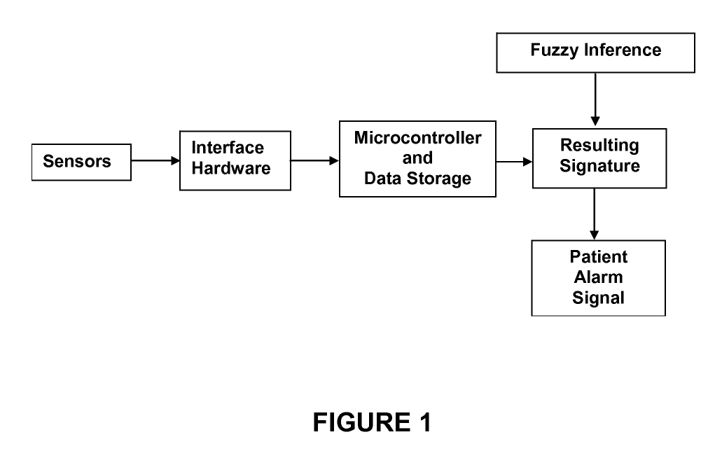

FIG. 1 shows a block diagram of one embodiment of the system. The information from sensors is transmitted through interface hardware to a microcontroller which may also contain data storage. The sensor information is converted into a resulting signature. A fuzzy inference is made on the signature, and based on the inference, a signal is generated. This signal can be an alarm for the patient, as shown in FIG. 1, or can be a signal transmitted to a doctor or caregiver, for example, or other examples as described herein.

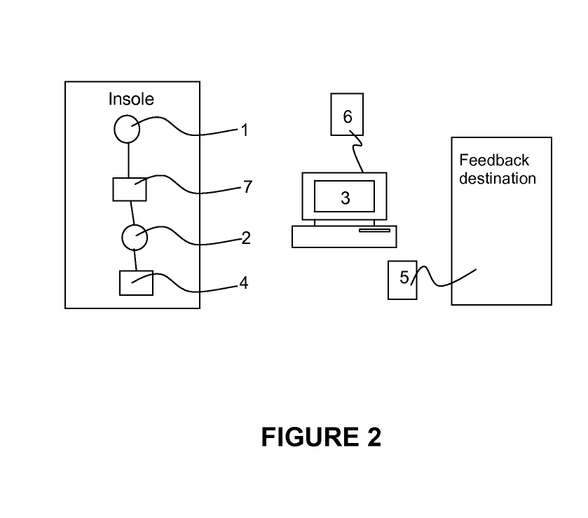

FIG. 2 illustrates a more detailed example of the system. In this example, the system comprises force/pressure sensors (1) (such as FlexiForce, Teskan), an integrated microcontroller/radio transmission and receiver communication system (2) (such as MICA2Adot Mote) and data collection software (3) (such as LabView). The compact sensors (four in one example) were placed in the insole pad of a shoe. The thin, flexible sensors measures force on various points on the foot (heel, toe, outer ball, inner ball). The signal is conditioned by a circuit (4) and prepared for evaluation and storage by a microcontroller system (2). Here all the algorithm calculations and stability alarm programming is stored and operated. Self learning algorithms are utilized to minimize the amount of on board stored data and provide the appropriate electrical stimulus to an audio/visual/wearable feedback system (5). The system transmits the packets of data for long term storage to a base station (laptop) (6) via a radio link. Measurement data can to down loaded via a directional antenna on the base station. The CC1000 radio (2) operates in the 900 MHz ISM band utilizing a Spread Spectrum Frequency Hopping scheme. This scheme divides the band into a series of sub-bands that the radio “hops” through an algorithmic manner. Only the radios communicating with each other know the “hopping” sequence. Thus interference can be avoided by hopping to different frequencies within the bank, and the system can operate within a multi radio (e.g., multi-patient) network. Small, replaceable batteries (7) power the insole unit, with unit lifetimes estimated to be 25 hours at full power. However, the insole sensor system goes to sleep when not in range of the base station or when the patient is not in motion, extending unit life times to weeks. As an additional feature to this invention, a piezo-based power generation can be used that converts the kinetic energy from the walking into electric power to make the system independent of any batteries. Data received at the base computer is decoded and stored for future use. Alarms are provided within the system when the algorithm detects a pending illness. As opposed to traditional gait collection systems, the insole device is designed to be convenient to use in environments outside of the lab as the small size and wireless module of the insole is user friendly and helps avoid distraction and maintain minimum interference with natural gait.

Once the data is received from sensors, an artificial intelligence algorithm (fuzzy interference) will be activated to analyze and develop a signature about the state of walking stability of the patient. If this signature falls within a stable region, then a GREEN light LED is turned on, or a certain frequency audio is activated. If the signature is outside of the stability region, then a RED light LED is turned on, or a different frequency audio is activated. In case that the signature falls within the fuzzy bands of stability/instability, then a YELLOW light LED is turned on, or a third alarm audio frequency would be activated.

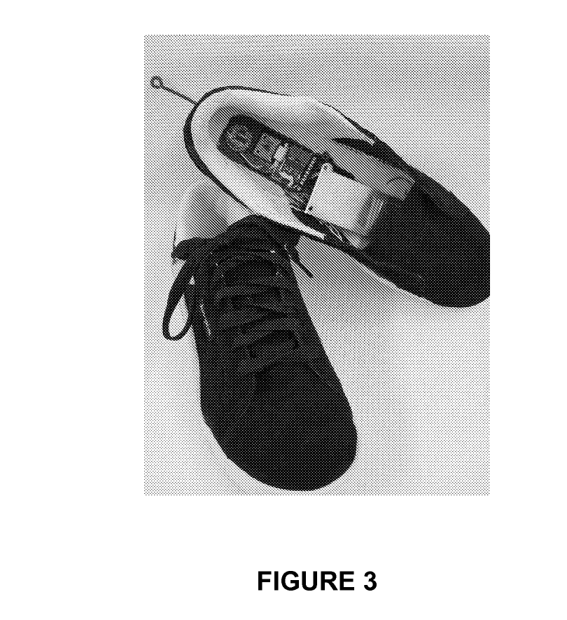

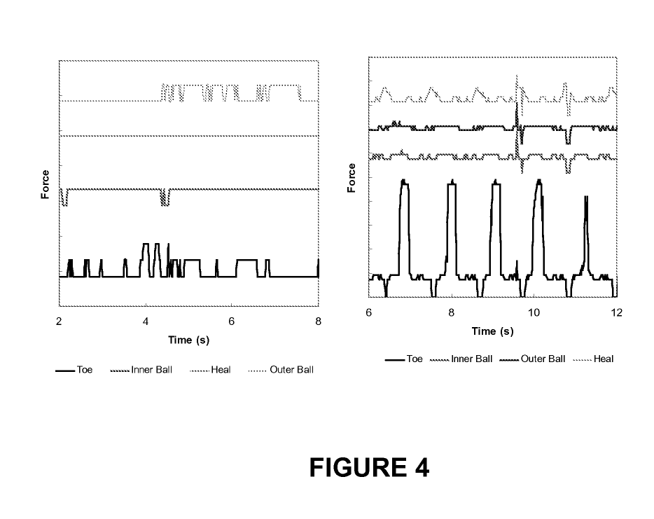

A device incorporating the system has been made and tested. FIG. 3 shows one example of a shoe containing the system described here. FIG. 4 shows exemplary data obtained from the example shoe. The plots in FIG. 4 show force versus time data for sensors measuring forces beneath the toe (bottom line), inner ball (second line from bottom), heel (third line from bottom) and outer ball (top line) as the subject balanced on a single foot (left plot) and normally walked (right plot). A wireless sneaker with embedded sensors and electronics has also been developed.

Although the description herein contains many specificities, these should not be construed as limiting the scope of the invention, but as merely providing illustrations of some of the embodiments of the invention. Thus, additional embodiments are within the scope of the invention and within the following claims. All references cited herein are hereby incorporated by reference to the extent that there is no inconsistency with the disclosure of this specification. Some references provided herein are incorporated by reference herein to provide details concerning additional methods of analysis and additional uses of the invention.

When a Markush group or other grouping is used herein, all individual members of the group and all combinations and subcombinations possible of the group are intended to be individually included in the disclosure. Every combination of components described or exemplified can be used to practice the invention, unless otherwise stated. One of ordinary skill in the art will appreciate that methods, device elements, and components other than those specifically exemplified can be employed in the practice of the invention without resort to undue experimentation. All art-known functional equivalents, of any such methods, device elements, and components are intended to be included in this invention. Whenever a range is given in the specification, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of elements of a device, is understood to encompass those methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. The definitions are provided to clarify their specific use in the context of the invention.

We claim:

1. A system for monitoring gait comprising:
(a) a pressure sensor;
(b) an algorithm which compares the data from the pressure sensor to a stability profile, and provides a feedback value;
(c) means for communicating the feedback value; and
(d) a power source.

2. The system of claim 1, wherein the means for communicating the feedback value is selected from the group consisting of: visual indication, tactile indication, audible indication and combinations thereof.

3. The system of claim 1, further comprising one or more accelerometers.

4. The system of claim 1, wherein the pressure sensor is located in a shoe, shoe insole, or sock.

5. The system of claim 1, further comprising a plurality of pressure sensors.

6. The system of claim 1, comprising pressure sensors at different parts of the foot.

7. The system of claim 1, comprising less than 10 pressure sensors.

8. The system of claim 1, comprising more than 2 pressure sensors.

9. The system of claim 1, wherein the power source is kinetic energy.

10. The system of claim 1, wherein the power source is alternating or direct current.

11. The system of claim 1, wherein the power source is one or more batteries.

12. A system for monitoring gait comprising:
(a) a plurality of sensors which generate a signal;
(b) a circuit means electrically connected to the plurality of sensors whereby said signal is collected;
(c) a transmission means to transmit the signal;
(d) a power source electrically connected to said plurality of sensors, circuit means, and transmission means;
(e) a software program that receives the transmitted signal and compares the transmitted signal to a stability profile and generates a feedback signal;
(f) a feedback means which transmits the feedback signal.

13. The system of claim 12, wherein the sensors comprise a plurality of pressure sensors and optionally one or more accelerometers.

14. The system of claim 12, wherein the feedback means is selected from the group consisting of: visual indication, tactile indication, audible indication and combinations thereof.

15. The system of claim 12, wherein the sensors are located in a shoe, shoe insole, or sock.

16. The system of claim 12, wherein the pressure sensors are located at different parts of the foot.

17. The system of claim 12, comprising less than 10 pressure sensors.

18. The system of claim 12, comprising more than 2 pressure sensors.

19. The system of claim 12, wherein the power source is kinetic energy.

20. The system of claim 12, wherein the power source is alternating or direct current.

21. The system of claim 12, wherein the power source is one or more batteries.

22. A method for gait analysis comprising:
collecting signals from one or more sensors, generating a test profile;
comparing the test profile to a stability profile;
generating a feedback signal;
communicating the feedback signal.

23. The method of claim 22, wherein the sensors comprise a plurality of pressure sensors located in pressure proximity to a patient's foot and optionally one or more accelerometers.

24. The method of claim 22, wherein the comparing step is performed by fuzzy logic.

25. The method of claim 22, wherein the communicating step is one or more of:
visual, tactile, and audible.
